SEQUENCE LISTING (2 sequences)

SEQ ID NO: 1 - protein, 40 aa, Artificial Sequence
  Synthetic polypeptide
GIGDPVTCLKSGAICHPVFCPRRYKQIGTCGLPGTKCCKK

SEQ ID NO: 2 - protein, 10 aa, Artificial Sequence
  Synthetic polypeptide
LVPRGSIEGR

(19) United States
(12) Patent Application Publication
(10) Pub. No.: US 2011/0300127 A1
(43) Pub. Date: Dec. 8, 2011

(54) DRUG-CONTAINING COMPOSITION

(75) Inventors: Kentaro Nakamura, Ashigarakami-gun (JP); Shouji Ooya, Ashigarakami-gun (JP); Tetsuo Hiratou, Ashigarakami-gun (JP)

(73) Assignee: FUJIFILM CORPORATION, Minato-ku, Tokyo (JP)

(21) Appl. No.: 12/994,707

(22) PCT Filed: May 27, 2009
(86) PCT No.: PCT/JP2009/002331

§ 371 Date: Nov. 24, 2010

(30) Foreign Application Priority Data
May 27, 2008 (JP) 2008-137924

(51) Int. Cl. A61K 47/42; A61K 39/395

(52) U.S. Cl. 424/130.1; 514/773; 514/774; 514/776

(57) ABSTRACT

It is an object of the present invention to provide a drug-containing composition capable of stably retaining a water-soluble compound without the loss of drug efficacy of a pharmaceutically active ingredient and thus preventing the compound from diffusing in the body, which is unlikely to cause disassociation at a non-target site and thus achieves disassociation/sustained release/delivery specifically at a target site. The present invention provides a composition which is composed of: (a) at least one a water-soluble compound; and (b) a carrier comprising a polymer having binding affinity with the water-soluble compound.

### TECHNICAL FIELD

The present invention relates to a drug carrier capable of stably retaining a water-soluble compound (preferably a pharmaceutically active ingredient) without the loss of drug efficacy of a pharmaceutically active ingredient and thus preventing the compound from diffusing in the body, which is unlikely to cause disassociation at a non-target site and thus achieves disassociation/sustained release/delivery specifically at a target site.

### BACKGROUND ART

It has been known that handleability of a water-soluble pharmaceutically active ingredient is generally good for in vivo administration such as oral administration or administration into blood. However, a water-soluble pharmaceutically active ingredient, by its nature, tends to diffuse so as to be quickly excreted from blood, which is problematic. In general, renal excretion of a substance of 5 nm or less in size is likely to take place.

A known method for increasing the stability of a water soluble pharmaceutically active ingredient in blood is a method involving pegylation or chemical modification of such active ingredient or a method using a liposome as a drug carrier (e.g., Patent Document 1). However, there is a concern that the use of such a method involving pegylation or chemical modification might result in the loss or reduction of original drug efficacy of a pharmaceutically active ingredient. In addition, in the case of a method using a liposome as a drug carrier, retention of a water-soluble drug is usually difficult. The following are known examples of a method for allowing a liposome to retain a water-soluble drug, in addition to the method disclosed in Patent Document 1:

(a) a reversed-phase evaporation method (Non-Patent Document 1);
(b) a method comprising subjecting a drug itself to chemical modification (Non-Patent
(c) a method using a different adjuvant or the like (Non-Patent Documents 5 and 6);
(d) a method comprising changing properties of a liposome or a liposome membrane itself (Non-Patent Document 7 and 8); and
(e) a method using a charged phospholipid that can form a pair with an oppositely charged drug (Non-Patent Documents 9 and 10 and Patent Document 2).
Each of the above is insufficient in terms of the retention rate.

In addition, even if a water-soluble drug can be retained, in one case, weak binding between a drug carrier and a pharmaceutically active ingredient often results in dissociation, and in other cases, sustained release of an encapsulated water-soluble drug cannot take place or does not take place in an appropriate manner. This is seriously problematic.

A specific example is described below. For instance, administration of a hydrophilic pharmaceutically active ingredient such as dopamine or serotonin into blood tends to result in renal excretion, causing reduction of stability in blood. However, there is a concern that pegylation or chemical modification of the active ingredient might cause the loss or reduction of original drug efficacy. It is difficult to achieve stable retention of the active ingredient with the use of a drug carrier such as a liposome. In addition, it is difficult to achieve targeting for sustained release in a specific manner at a target receptor site in the body by a known method, which is problematic.

Meanwhile, in recent years, as a method for solubilizing a poorly water-soluble drug, a method for solubilizing such drug with serum albumin has been used (Patent Document 3). However, since water-soluble compounds have low binding affinity to serum albumin, it has been impossible to use such technique in practice.

### Prior Art Documents

### Non-Patent Documents

### Non-Patent Document 1: Proceedings of the National Academy of Sciences, USA, vol. 75, p. 4194, 1978

### Non-Patent Document 2 : International Journal of Pharmaceutics, vol. 14, p. 191, 1983

### Non-Patent Document 3 : Journal of Pharmacobio Dynamics, vol. 7, p. 120, 1984

### Non-Patent Document 4 : Chemical and Pharmaceutical Bulletin, vol. 36, p. 3574, 1988

### Non-Patent Document 5 : Journal of Pharmaceutical Sciences, vol. 71, p. 958, 1982

### Non-Patent Document 6 : Drug Development and Industrial Pharmacy, vol. 10, p. 613, 1984

### Non-Patent Document 7 : Biochimica et Biophysica Acta, vol. 812, p. 66, 1985

### Non-Patent Document 8 : Biochimica et Biophysica Acta, vol. 857, p. 123, 1986

### Non-Patent Document 9 : Biochemical and Biophysical Research Communications, vol. 107, p. 136, 1982

### Non-Patent Document 10: International Journal of Pharmaceutics, vol. 17, p. 135, 1983

### Patent Documents

### Patent Document 1 : JP Patent Publication (Kokai) No. 5-194191 A (1993)

### Patent Document 2 : JP Patent Publication (Kokai) No. 62-87514 A (1987)

### Patent Document 3 : JP Patent Publication (Kohyo) No. 2000-508806 A

### SUMMARY OF THE INVENTION

### Problem to be Solved by the Invention

It is an object of the present invention to provide a drug-containing composition capable of stably retaining a water-soluble compound without the loss of drug efficacy of a pharmaceutically active ingredient and thus preventing the compound from diffusing in the body, which is unlikely to cause disassociation at a non-target site and thus achieves disassociation/sustained release/delivery specifically at a target site.

### Means for Solving Problem

As a result of intensive studies in order to attain the above object, the present inventors found that the concentration of a water-soluble drug in blood can be maintained for many hours by using, as a drug carrier for transporting a water-soluble drug, a biopolymer having a high level of binding affinity to such drug. The finding has led to the completion of the present invention.

Thus, the present invention provides a composition which is composed of: (a) at least one a water-soluble compound; and (b) a carrier comprising a polymer having binding affinity with the water-soluble compound.

Preferably, the polymer having binding affinity with the water-soluble compound is a polymer having binding affinity that is a dissociation constant Kd of 10⁻³ to 10⁻¹⁵ M with the water-soluble compound.

Preferably, the water-soluble compound is a pharmaceutical product.

Preferably, the polymer having binding affinity with the water-soluble compound is a protein.

Preferably, the protein is: a protein containing an amino acid sequence of a receptor of a water-soluble compound, a sequence responsible for binding which is contained in a receptor of a water-soluble compound, an amino acid sequence of an antibody to a water-soluble compound, or a sequence responsible for binding which is contained in an antibody to a water-soluble compound; a lectin protein; or a protein containing a sequence responsible for binding with a water-soluble compound which is contained in a lectin protein; a protein that binds to a water-soluble compound; or a protein containing a sequence responsible for binding which is contained in a protein that binds to a water-soluble compound.

Preferably, the protein is a protein which was produced by gene recombinant techniques.

Preferably, a different protein is further bound directly or via a linker to the N-terminal and/or the C-terminal of the protein.

Preferably, the different protein binding to the N-terminal and/or the C-terminal of the protein is a protein that can control the release of a water-soluble compound by causing a steric hindrance or a protein that functions in vivo as a scaffold.

Preferably, the protein that functions in vivo as a scaffold is gelatin, collagen, albumin, elastin, or fibrin.

Preferably, the composition of the present invention is a pharmaceutical composition for administering the water-soluble compound to patients.

### Effects of the Invention

The concentration of a water-soluble drug in blood can be maintained for many hours with the use of the drug-containing composition of the present invention.

### BRIEF DESCRIPTION OF THE DRAWINGS

FIG. 1 shows a representative example of the structure of the composition of the present invention.

FIG. 2 shows results of measurement of blood serotonin concentration.

### EMBODIMENT FOR CARRYING OUT THE INVENTION

Hereinafter, the present invention is described in detail. The composition of the present invention is characterized in that it is composed of: (a) at least one a water-soluble compound; and (b) a carrier comprising a polymer having binding affinity with the water-soluble compound. The term “binding affinity” used herein refers to, for example, a specific non-covalent binding interaction, such as an enzyme-substrate, ligand-receptor, or enzyme-coenzyme interaction, which is susceptible to competitive inhibition caused by an adequate competitive molecule. In the present invention, the dissociation constant Kd for the binding between a water-soluble compound and a carrier is preferably 10⁻³ to 10⁻¹⁵ M, more preferably 10⁻⁵ to 10⁻¹³ M.

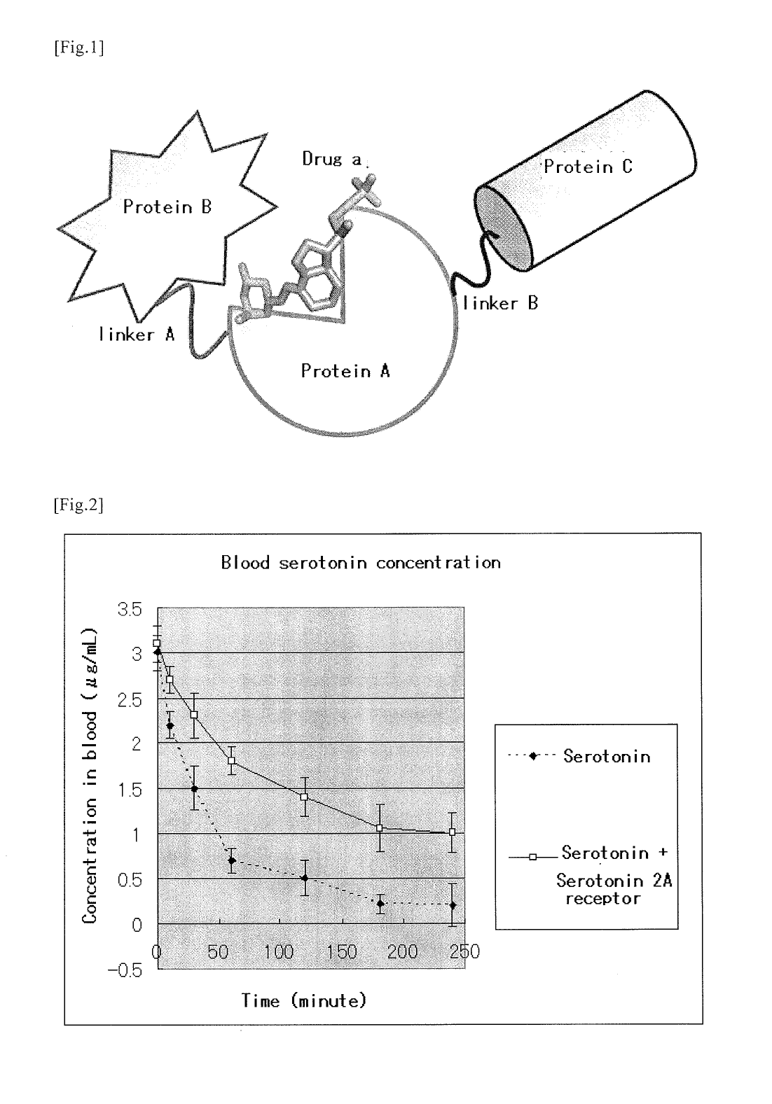

FIG. 1 shows a representative example of the structure of the composition of the present invention. Drug “a” (water-soluble compound), protein A (a polymer having binding affinity to a water soluble compound), protein B, protein C, linker A, and linker B shown in FIG. 1 are described below.

<Drug “a”>

The term “water-soluble compound” used herein refers to a compound (and particularly, a pharmaceutical compound) with a solubility in water (1 mL) determined at 25° C. of 10 μg to 700 mg, desirably 100 μg to 500 mg, and most desirably 1 mg to 300 mg. It may be possible for such compound not to be dissolved at 25° C. if it can be dissolved at a high temperature of 100° C. and remain dissolved after being cooled to 25° C. In addition, the pH of water used for solubility determination is specifically 6 to 8.5, preferably 6.5 to 7.5, and most preferably 7.0.

More preferably, the pharmaceutical compound has a Log P of −10 to 2 and more preferably −10 to 1, provided that a relevant water soluble pharmaceutically active ingredient satisfies the above solubility conditions. In general, Log P is used as an indicator of the hydrophilicity/hydrophobicity of a compound. The log (Log P) of the 1-octanol/water partition coefficient obtained by a flask shaking method (buffer solution: pH 7.4) has been widely used. It is also possible to obtain such value by calculation instead of actual measurement. (LogP used herein is calculated by the CLOGP program for the Hansch-Leo fragmental method, which is included in the “PCModels” system (Daylight Chemical Information Systems).)

The drug is a physiologically active ingredient. Specific examples of such drug include analgesic agents, anesthetic agents, anti-anginal agents, antiarthritic agents, anti-arrhythmic agents, antiasthmatic agents, antibiotic agents, anticancer agents, anticholinergic agents, anticoagulants, anticonvulsants, antidepressants, antidiabetic agents, antifungal agents, antiglaucoma agents, antigout agents, antiparasitic agents, antihistamines, antihyperlipidemic agents, antihypertensive agents, antiinflammatory agents, antimalarial agents, antimigraine agents, antimuscarinic agents, antinauseants, anti-obesity agents, anti-osteoporosis agents, antipanic agents, antiparkinsonism agents, antiprotozoal agents, antipruritics, antipsychotic agents, antipyretics, antitubercular agents, antitussive agents, antiulcer agents, antiviral agents, anxiolytics, appetite suppressants, calcium channel antagonists, cardiac inotropic agents, β blockers, bone density regulators, central nervous system stimulants, cognition enhancers, corticosteroids, decongestants, diuretics, gastrointestinal agents, genetic materials, hormonolytics, hypnotics, hypoglycemic agents, immunosuppressants, keratolytics, leukotriene inhibitors, macrolides, mitotic inhibitors, muscle relaxants, narcotic antagonists, neuroleptic agents, nicotine, parasympatholytic agents, peptides, polypeptides, proteins, saccharides, sedatives, sex hormones, sympathomimetic agents, tocolytics, tranquilizers, vasodilators, vitamins, psychotropic agents, and combinations thereof.

For example, the following drugs can be used: fosphenytoin disodium, irinotecan hydrochloride hydrate, omeprazole sodium, fludarabine phosphate, docetaxel hydrate, peginterferon-α-2a(recombinant), micafungin sodium, adefovir dipivoxil, levofloxacin hydrate, cytarabine, ramatroban, fluvastatin sodium, sinecatechins, fosamprenavir calcium, belotecan hydrochloride, esomeprazole magnesium, timolol maleate (Wakamoto), nelarabine, dexketoprofen, quinupristin/dalfopristin, bepotastine besilate, melevodopa, etoposide phosphate, topotecan hydrochloride, methylprednisolone suleptanate, trandolapril, colforsin daropate hydrochloride, Fiber SS, epinastine hydrochloride, nitroglycerin(Sanwa Kagaku), diclofenac sodium(Wakamoto), pranlukast hydrate, fosfomycin tromethamine, lercanidipine hydrochloride, rimexolone, nicardipine hydrochloride, sodium prasterone sulfate, canrenoate potassium, mitotane, ranimustine, insulin, dexamethasone palmitate, budesonide, menatetrenone, bucladesine sodium, satraplatin, fospropofol disodium, ceftobiprole medocaril, ceftizoxime alapivoxil hydrochloride, ceftaroline fosamil, ulipristal acetate, isavuconazonium chloride, becatecarin tartrate, talotrexin ammonium, retaspimycin, triciribine phosphate, amonafide dihydrochloride, Triapine, motexafin lutetium, lurtotecan dihydrochloride, iganidipine, gluconate potassium (Kowa), repagermanium, ataprost alfadex, and exatecan mesilate.

Also, a variety of nucleic acid drugs can be used as water-soluble drugs. For instance, siRNA, aptamer, antisense RNA, decoy DNA, and DNA for gene therapy can be used. Preferable examples of an anticancer agent used as a water-soluble drug include cisplatin, 5-FU, bleomycin, mitomycin, epirubicin hydrochloride, ifosfamide, carboplatin, etoposide, cyclophosphamide, and doxorubicin. Preferable examples of an antipsychotics/antipanic agent/antiparkinsonism agent/an agent for central nervous disease used as a water-soluble drug include serotonin, dopamine, and L-dopa. Preferably, water-soluble psychotropic agents can also be used as water-soluble drugs. For example, Amobarbital salt, flunitrazepam, clorazepate dipotassium, phenobarbital, and pemoline can be used.

In one embodiment of the present invention, a water-soluble drug is an anti-inflammatory agent, which is generally a non-steroidal anti-inflammatory agent (NSAID) or a COX-2 inhibitor. Specific examples of such drugs include, without limitation, acetylsalicylic acid, alclofenac, alminoprofen, benoxaprofen, butibufen, bucloxic acid, carprofen, celecoxib, clidenac, diclofenac, diflunisal, etodolac, fenbufen, fenoprofen, fentiazic, flufenamic acid, flufenasol, flurbiprofen, furofenac, ibufenac, ibuprofen, indomethacin, indoprofen, isoxepac, isoxicam, ketoprofen, ketorolac, meclofenamic acid, mefenamic acid, meloxicam, miroprofen, naproxen, oxaprozin, oxyphenbutazone, oxpinac, parecoxib, phenylbutazone, piclamilast, piroxicam, pirprofen, pranoprofen, rofecoxib, sudoxicam, sulindac, suprofen, tenclofenac, tiaprofenic acid, tolfenamic acid, tolmetin, tramadol, valdecoxib, zomepirac, and pharmacologically active basic addition salts thereof.

In another embodiment of the present invention, a water-soluble drug is a bisphosphonic acid derivative useful in the diagnosis and treatment of diseases and symptoms related to bone resorption, calcium metabolism, and phosphate metabolism. Examples of such bisphosphonic acid include 1-hydroxyethane-1,1-diphosphoric acid (etidronic acid), 1,1-dichloromethylene-1,1-bisphosphonic acid (clodronic acid), 3-amino-1-hydroxypropylidyne-1,1-bisphosphonic acid (pamidronic acid), 4-amino-1-hydroxybutylidene-1,1-bisphosphonic acid (alendronic acid), 6-amino-1-hydroxyhexylidene-1,1-bisphosphonic acid (neridronic acid), (4-chlorophenyl)thiomethane-1,1-diphosphoric acid (tiludronic acid), 1-hydroxy-2-(3-pyridinyl)-ethylidene-1,1-bisphosphonic acid (risedronic acid), cycloheptylaminomethylene-1,1-bisphosphonic acid (cimadronic acid), 1-hydroxy-3-(N-methyl-N-pentylamino)propylidyne-1,1-bisphosphonic acid (ibandronic acid), 3-(dimethylamino)-1-hydroxypropylidyne-1,1-bisphosphonic acid (olpadronic acid), [2-(2-pyridinyl)ethylidene]-1,1-bisphosphonic acid (piridronic acid), and 1-hydroxy-2-(1H-imidazole-1-yl)ethylidene-1,1-bisphosphonic acid (zoledronic acid).

### <Protein A>

Protein A (a polymer having binding affinity with a water-soluble compound) is a protein having affinity with Drug “a”. A receptor, a target protein, or a binding protein of Drug “a” can be used. Examples thereof include a vitamin D3 receptor, HMG-CoA reductase, an ADP receptor (P2Y12), a type-L calcium channel, a proton pump, a serotonin receptor, a dopamine receptor, a dopamine D2 receptor, an angiotensin II receptor, a melatonin MT1/MT2 receptor, an α2δ subunit of voltage-dependent calcium channel, PDGFR-α, PDGFR-β, VEGFR1, VEGFR2, VEGFR3, KIT, FLT3, CSF-1R, RET, a ribosome 50S subunit, Tubulin, DNA helicase, RNA polymerase, an acetylcholine receptor, a G protein conjugated receptor, a muscarinic acetylcholine receptor, an adenosine receptor, an adrenaline receptor, a GABA receptor (type B), an angiotensin receptor, a cannabinoid receptor, a cholecystokinin receptor, a glucagon receptor, a histamine receptor, an olfactory receptor, an opioid receptor, rhodopsin, an secretin receptor, a somatostatin receptor, a gastrin receptor, an erythropoietin receptor, an insulin receptor, a cell growth factor receptor, a cytokine receptor, a guanylate cyclase receptor, a GC-A, GC-B, or GC-C guanylin receptor, a nicotinic acetylcholine receptor, a glycine receptor, a GABA receptor (type A or C), a glutamic acid receptor, a type-3 serotonin receptor, inositol triphosphate (IP3) receptor, ryanodine receptor, a steroid hormone receptor, a sex hormone (androgen, estrogen, or progesterone) receptor, a vitamin D receptor, a glucocorticoid receptor, a mineralocorticoid receptor, a thyroid hormone receptor, a retinoid receptor, a peroxisome proliferator-activated receptor (PPAR), an insect molting hormone (ecdysone) receptor, a dioxin receptor (AhR), and a benzodiazepine receptor.

Protein A may be a naturally occurring biologically derived protein or a protein produced by gene recombination technology. However, with regard to the designing described below, a protein produced by gene engineering is preferable. Such protein may comprise a naturally occurring sequence or a sequence newly designed depending on application. As a sequence newly designed depending on application, a sequence substantially responsible for binding extracted from a naturally derived sequence of the protein, which is directly or indirectly essential for the binding to Drug “a” can be used. In addition, as a newly designed sequence, a sequence obtained by partially altering the amino acid sequence contained in a natural sequence of the protein can be used. Specifically, an amino acid sequence of the protein or an amino acid sequence contained in a sequence responsible for binding extracted from the protein can be adjusted, thereby adjusting the solubility of the protein or interaction between the protein and a different biologically derived molecule. In addition, a side chain that is contained in a sequence responsible for the binding to Drug “a” and is directly or indirectly involved in the binding to Drug “a” can be substituted with a different side chain, thereby attenuating or intensifying the affinity. Such substitution can be carried out in a manner such that the protein sequence is partially altered or 1 to 50 residue(s) are newly inserted into or deleted from the protein sequence.

Further, the above protein may be chemically modified in vivo or in vitro. For instance, chemical modification of amino groups in the protein that can be carried out includes, but is not limited to, formation of guanidyl, amidin, or reduced alkyl, carbamylation, acetylation, succinylation, maleylation, acetoacetylation, formation of nitrotroponyl, deaminization, modification with a carbonyl compound, dinitrophenylation, and/or trinitrophenylation. In addition, chemical modification of carboxyl groups contained in the protein that can be carried out includes, but is not limited to, amidation and/or esterification. Further, for chemical modification, modification with sugar chains may be carried out.

Furthermore, the above protein may contain an auxiliary molecule that allows the three-dimensional structure to be maintained, the ability to bind to a ligand or substrate to be secured, or the in vivo stability or physiological functions to be maintained. Examples of such auxiliary molecules that can be used include Zn, Fe, Cd, Cu, Au, Ag, Pt, Hg, Na, Cl, K, Ca, Li, Mg, Al, Co, Mn, Cr, Ga, Ge, Ni, Br, Rb, Mo, and

Pb atoms or molecules, complexes (e.g., heme and protoheme complexes) comprising such atoms or molecules, and ions or complex ions thereof. In addition, a coenzyme, an electron carrier, or the like can be used as such auxiliary molecule. Specific examples thereof include, but are not limited to, quinone, pyrroloquinoline quinone, topaquinone, tryptophan-tryptophylquinone, lycine tyrosyl quinone, cystenyl-tryptophanquinone, thiamine diphosphate, coenzyme A (pantothenic acid), coenzyme R (biotin), coenzyme F (folic acid), ATP (adenosine triphosphate), uridine diphosphate glucose, NAD⁺/NADH (nicotinamide adenine dinucleotide), FMN/FMNH₂ (flavin mononucleotide), FAD/FADH₂ (flavin adenine dinucleotide), ubiquinone, cytochrome, NADP⁺/NADPH (nicotinamide adenine dinucleotide phosphate), plastoquinone, plastocyanin, ferredoxin, chlorophyll, pheophytin, thioredoxin, menaquinone, caldariellaquinone, coenzyme F₄₂₀, rhodoquinone, Riske, and Blue-Cu.

### <Protein B>

A different protein (namely, Protein B) can be bound to Protein A.

A variety of structural proteins or structural peptides can be used as Protein B that can be bound to Protein A. For instance, Protein B can regulate the release of Drug “a” by causing a steric hindrance. Specifically, in order to control the rate of release of Drug “a” from the sequence domain responsible for binding or the proportion of released Drug “a”, a different structural protein sequence that can serve as a “cap” in the steric structure (hereinafter referred to as “cap protein sequence”) can be used as Protein B. That is, it is possible to design a sequence that can serve as a cap in the three-dimensional structure and to use such sequence with Protein A. In addition, examples of such cap protein sequence that can be used include GIGDPVTCLKSGAICHPVFCPRRYKQIGTCGLPGTKCCKK (each letter denoting a single amino acid). Also, a protein sequence having unique functions can be used as Protein B. Such Protein B having unique functions can be modified depending on application and is not particularly limited. For example, a sequence having a function to exhibit antibacterial activity, blood sugar regulatory activity, activity of regulating the urge to eat, blood pressure regulatory activity, analgesic activity, antiviral activity, anticoagulating activity, vasoconstriction/vasodilatation activity, tranquilizing activity, antidepressive activity, mental exaltation activity, or adhesion activity can be used. More specific examples of such sequence include antibacterial peptides, defensin, lactoferricin, magainin, tachyplesin, angiotensin, bradykinin, T kinin, fibrinopeptides, natriuretic peptides (for atrial or cerebral natriuresis), urodilatin, guanine, uroguanine, endothelin, big endothelin, salusin, urotensin, oxytocin, vasopressin, neurophysin, proopiomelanocortin-derived peptides, posterior pituitary hormone, adrenocorticotropic hormone, corticotropin-like intermediate-lobe peptide, endorphin, lipotropin, melanocyte-stimulating hormone, hypothalamic hormone, urocortin, somatostatin, cortistatin, TRH, prolactin, pituitary adenylate cyclase-activating peptide, metastin, tachykinin, substance P, neuropeptide, neurokinin, endokinin, neurotensin, neuromedin, ghrelin, obestatin, agouti-related protein , melanin-concentrating hormone, neuropeptide, orexin, opioidpeptide, dynorphin, neoendorphin, leumorphin, methionine enkephalin, leucine enkephalin, methionine enkephalin, adrenorphin, endomorphin, nociceptin, orphanin, nocistatin, RFamide peptide, galanin, gastrin, cholecystokinin, motilin, pancreatic polypeptide, gastric inhibitory peptide, peptide YY, peptide HM, vasoactive intestinal peptide, secretin, apelin, insulin, C peptide, insulin-like peptide, relaxin, relaxin-like peptide, glucagon, glicentin, glucagon-like peptide, oxyntomodulin, CGRP, adrenomedullin, proadrenomedullin, calcitonin receptor-stimulating peptide, amyrin, calcitonin, catacalcin, parathyroid hormone, cathelicidin, thymosin, and humanin.

In addition, in order to allow Protein B to pass through the blood-brain barrier, a peptide such as microglia-derived brain transfer polypeptide sequence described in WO2005/014625 (International Application No.: PCT/JP2004/011668) that can pass through the blood-brain barrier can be used as Protein B. Protein A and Protein B may be directly bound to each other, or they may be bound to each other via a linker (hereinafter referred to as Linker A). Although an object of “targeting to a target site after transition to blain” is present in the aforementioned technique, this object can also be solved by the use of the present invention.

Linker A is not particularly limited, as long as it binds Protein A and Protein B. Preferably, a versatile linker sequence or a linker designed for specific purposes can be used in the form of a protein sequence containing peptide bonds. As a versatile linker, a peptide comprising 2 to 40 residues can be used. In order to obtain a linker designed for a specific purpose, a linker can be designed in accordance with such purpose and is not particularly limited. However, a sequence that is cleaved in vivo in the presence of protease activity, a sequence that is phosphorylated by a certain factor, a sequence that is hydrolyzed, a sequence containing a sequence to be methylated, or the like can be used. More specifically, a sequence that is cleaved by a blood-clotting factor protease or a sequence that is cleaved by a matrix metalloprotease can be used. However, the above linker is not limited to such examples. As examples of a sequence that is cleaved by thrombin, the sequences described in the following can be used: Thrombin specificity, Requirement for apolar amino acids adjacent to the thrombin cleavage site of polypeptide substrate, Jui-Yoa CHANG. Eur. J. Biochem. 151, 217-224 (1985) FEBS (Factor Xa, prothrombin, or FactorVII); and X-ray Structure of Active Site-inhibited Clotting Factor Xa, IMPLICATIONS FOR DRUG DESIGN AND SUBSTRATE RECOGNITION, Hans Brandstetter, et. al. Volume 271, Number 47, Issue of Nov. 22, 1996 pp. 29988-29992, THE JOURNAL OF BIOLOGICAL CHEMISTRY. For example, the sequence LVPRGSIEGR (each letter denoting a single amino acid) can be used.

### <Protein C>

A different protein, namely Protein C, can be bound to Protein A or Protein B described above.

A variety of structural proteins and structural peptides can be used as Protein C. For instance, a protein sequence that functions in vivo as a scaffold can be designed and used. Protein C is not limited as long as it is a protein that can function as a scaffold. Examples of Protein C that can be used include gelatin, collagen, albumin, elastin, and fibrin. In addition, Protein C may be a natural biologically derived substance or a gene recombinant.

Protein C may be bound directly or via a linker (hereinafter referred to as Linker B) to Protein A or Protein B.

Linker B is not particularly limited, as long as it binds Protein A (or Protein B) and Protein C. Preferably, a versatile linker sequence or a linker designed for specific purposes can be used in the form of a protein sequence containing peptide bonds. As a versatile linker, a peptide comprising 2 to 40 residues can be used. In order to obtain a linker designed for a specific purpose, a linker can be designed in accordance with such purpose and is not particularly limited. However, a sequence that is cleaved in vivo in the presence of protease activity, a sequence that is phosphorylated, a sequence that is hydrolyzed, a sequence containing a sequence to be methylated, or the like can be used. More specifically, a sequence that is cleaved by a blood-clotting factor protease or a sequence that is cleaved by a matrix metalloprotease can be used. However, the above linker is not limited to such examples. As examples of a sequence that is cleaved by thrombin, the sequences described in the following can be used: Thrombin specificity, Requirement for apolar amino acids adjacent to the thrombin cleavage site of polypeptide substrate, Jui-Yoa CHANG. Eur. J. Biochem. 151, 217-224 (1985) FEBS (Factor Xa, prothrombin, or FactorVII); and X-ray Structure of Active Site-inhibited Clotting Factor Xa. IMPLICATIONS FOR DRUG DESIGN AND SUBSTRATE RECOGNITION, Hans Brandstetter, et. al. Volume 271, Number 47, Issue of Nov. 22, 1996 pp. 29988-29992, THE JOURNAL OF BIOLOGICAL CHEMISTRY. For example, the sequence LVPRGSIEGR (each letter denoting a single amino acid) can be used.

Known methods can be used to cause the expression of the proteins described above and to produce such proteins.

The use of the composition of the present invention is not particularly limited. However, the composition can be used for therapeutic drugs for a variety of diseases, and therefore it can be used as a topical therapeutic agent, an oral therapeutic agent, a parenteral injection, or the like.

The present invention is hereafter described in greater detail with reference to the following examples, although the present invention is not limited thereto.

### EXAMPLES

### Example 1

The experiment described below was conducted using, as a water-soluble drug model, serotonin (5-hydroxytryptamine, 5-HT) which is used as an agent for central nervous disease.

A human 5-hydroxytryptamine (serotonin) 2A receptor (NCBI sequence No. NP_{—}000612) was expressed as a His-tag fusion protein with the use of Escherichia coli BL21 (DE3) Codon-plus (and a vector (pQE30 Xa; QIAGEN)). For culture, an LB (Luria-Bertani) medium containing 100 μg/ml ampicillin was used. Preculture was carried out with the use of a 300-mL LB medium contained in a 500-mL Erlenmeyer flask at 37° C. Then, for main culture, 30 mL of the preculture solution was added to a 1.5-L LB medium (containing 100 μg/ml ampicillin) contained in a 3-L baffled Erlenmeyer flask and subjected to shake culture at 37° C. so as to result in 0.6 OD600. Thereafter, IPTG was added thereto to a final concentration of 0.5 mM for expression induction, followed by overnight shake culture at 30° C. Subsequently, cells were collected by centrifugation and washed. The obtained bacterial cells were suspended in a mixture of 200 mM NaCl, 50 mM sodium phosphate buffer, and 10 mM imidazole (pH 8.0), followed by ultrasonic disintegration for 5 minutes and centrifugation at 44,200 ×g for 30 minutes. Thus, the supernatant was obtained. The obtained supernatant was introduced at a flow rate of 0.1 ml/min into an Ni-column (Ni-NTA His-Bind Resin: Novagen; column volume: 50 ml) that had been preliminarily equilibrated with a solution A (300 mM NaCl, 50 mM sodium phosphate buffer, 20 mM imidazole, pH 8.0) for immobilization. The column was washed with 500 ml of a solution B (300 mM NaCl, 50 mM sodium phosphate buffer, 20 mM imidazole, pH 8.0), followed by elution with a solution C (300 mM NaCl, 50 mM sodium phosphate buffer, 250 mM imidazole, pH 8.0). Further, the eluate was subjected to gel filtration chromatography (with the use of a Superdex 75 10/300 GL column (GE); buffer: solution A) with the use of AKTA FPLC. High-purity fractions were exclusively collected, followed by dialysis/concentration. Eventually, a His-tag fusion serotonin 2A receptor protein dissolved in the final solution A was obtained.

Further, a step of cleaving the tag with FactorXa was carried out. Thus, the untagged serotonin 2A receptor protein was obtained using an Ni column.

A sufficient amount of the obtained serotonin 2A receptor protein was allowed to bind to serotonin. The resultant was administered to mice via intravenous injection. Then, changes in the blood serotonin concentration were quantified. Separation of serotonin from blood was carried out by column chromatography using an ion exchanger, followed by quantification by a fluorescence method. Quantification was carried out by the method of Yoshida A. et al., Br. J. Pharmacology. 2002; 137: 146-152. Here, the administration dose of serotonin alone and that of serotonin binding to a serotonin 2A receptor were equivalent in terms of the serotonin content used for experimentation. In addition, the serotonin concentration in mice to be used was preliminarily determined under usual conditions. A group of mice whose blood serotonin levels fell within a range of 200 ng/mL±50 ng/mL under usual conditions was used. FIG. 2 shows the results. By the use of the configuration of the present invention, the concentration of the water soluble drug in blood was successfully maintained for many hours.

1. A composition which is composed of: (a) at least one a water-soluble compound; and (b) a carrier comprising a polymer having binding affinity with the water-soluble compound.

2. The composition according to claim 1, wherein the polymer having binding affinity with the water-soluble compound is a polymer having binding affinity that is a dissociation constant Kd of 10⁻³ to 10⁻¹⁵ M with the water-soluble compound.

3. The composition according to claim 1, wherein the water-soluble compound is a pharmaceutical product.

4. The composition according to claim 1, wherein the polymer having binding affinity with the water-soluble compound is a protein.

5. The composition according to claim 4, wherein the protein is: a protein containing an amino acid sequence of a receptor of a water-soluble compound, a sequence responsible for binding which is contained in a receptor of a water-soluble compound, an amino acid sequence of an antibody to a water-soluble compound, or a sequence responsible for binding which is contained in an antibody to a water-soluble compound; a lectin protein; or a protein containing a sequence responsible for binding with a water-soluble compound which is contained in a lectin protein; a protein that binds to a water-soluble compound; or a protein containing a sequence responsible for binding which is contained in a protein that binds to a water-soluble compound.

6. The composition according to claim 4, wherein the protein is a protein which was produced by gene recombinant techniques.

7. The composition according to claim 4, wherein a different protein is further bound directly or via a linker to the N-terminal and/or the C-terminal of the protein.

8. The composition according to claim 7, wherein the different protein binding to the N-terminal and/or the C-terminal of the protein is a protein that can control the release of a water-soluble compound by causing a steric hindrance or a protein that functions in vivo as a scaffold.

9. The composition according to claim 8, wherein the protein that functions in vivo as a scaffold is gelatin, collagen, albumin, elastin, or fibrin.

10. The composition according to claim 1, which is a pharmaceutical composition for administering the water-soluble compound to patients.
